(19) United States
(12) Patent Application Publication
(10) Pub. No.: US 2020/0098469 A1
(43) Pub. Date: Mar. 26, 2020

(54) System, Method, and Apparatus for Mobile Medication Nurse

(71) Applicant: AVERLENT CORPORATION, Clearwater, FL (US)

(72) Inventor: Bruce Pitcher, Clearwater, FL (US)

(73) Assignee: AVERLENT CORPORATION, Clearwater, FL (US)

(21) Appl. No.: 16/695,958

(22) Filed: Nov. 26, 2019

Related U.S. Application Data
(63) Continuation-in-part of application No. 16/014,149, filed on Jun. 21, 2018.
Application No. 16/014,149 is a continuation-in-part of application No. 15/003,125, filed on Jan. 21, 2016.

(51) Int. Cl. G16H 40/20; G16H 20/10; G06Q 20/26; H04W 4/029

(52) U.S. Cl. CPC G16H 40/20; H04W 4/029; G06Q 20/26; G16H 20/10

(57) ABSTRACT

A system and method for dispatching a nurse for administering medications includes visits that are either scheduled or unscheduled. Scheduled visits occur on a regular basis and are assigned to a particular nurse, while unscheduled visits are assigned to an available nurse based upon, for example, the location of that nurse with respect to that of the patient. Upon completion of administering the medication, the nurse provides data (e.g. feedback) of the visit, including an indication of any medication that was not taken and why. In some embodiments, if any medication was not taken, information regarding such is forwarded to the physician that prescribed that medication for remedial action.

### CROSS-REFERENCE TO RELATED APPLICATION

This application is a continuation-in-part of U.S. patent application Ser. No. 16/014,149, filed Jun. 21, 2018 and is a continuation-in-part of U.S. patent application Ser. No. 15/003,125, filed Jan. 21, 2016, the disclosure of which are hereby incorporated by reference.

### FIELD

This invention relates to medical services and more particularly to a system for managing the administration of medications.

### BACKGROUND

As the national population ages, more and more people are seeking in-home services and/or assisted transportation to/from appointments, shopping, etc. Many in this population have what is known as advocates such as a family member (adult child, sibling, etc.) who is concerned about the care being delivered. It is not always possible for these advocates to be at the patient's location during the time when medication needs to be administered. Therefore, the patient is often responsible for self-administering of medications.

Unfortunately, many individuals, particularly those advanced in years or stricken with certain ailments, lack the skills and/or discipline to administer their own medication on a reliable basis. This is often frustrating for the patient as well as the patient's advocates and, in some situations, results in complications caused by missed medications. Many patients with Alzheimer's, dementia, or other afflictions are totally incapable of self-administration of medications.

When such patients have significant issues they either must move in with loved ones or be admitted to an assisted care facility. At the assisted care facility, various aides are available (e.g. nurses) to make sure medications are administered.

For those who have some issues, but not so significant that they must be admitted to an assisted care facility, some level of independent living is possible, but as many grow older or as certain illnesses progress, those people begin to have difficulty with self-administration of medications. Also, for some, medication administration is not viable due to the type of administration (e.g. by injection) or due to mobility issues with the patient.

Therefore, there are two classes of patients; those who need daily assistance with administration of medication and those who are capable of self-administration of medications, but tend to forget to take their medications at the prescribed time.

What is needed is a system that will manage a plurality of nurses (e.g. nurses) that are able to (or licensed to) administer medications to patients.

### SUMMARY

A system for managing a team of mobile medication nurses provides tools for scheduled visitations when medications need be administered to a patient as well as unscheduled visitations when there is a lack of acknowledgement that a specific medication was self-administered by the patient at the expected time. Tools are provided for scheduling the scheduled visits as well as tools for the mobile medication nurse to indicate they are available for unscheduled visits and, should there be a lack of such acknowledgement, one member of the mobile medication nurse team that has indicated that they are available and is local to the patient that failed to acknowledge self-medication is dispatched to make an unscheduled visit to that patient. Billing is performed automatically.

The system and method for dispatching a nurse for administering medications includes visits that are either scheduled or unscheduled. Scheduled visits occur on a regular basis and are assigned to a particular nurse, while unscheduled visits are assigned to an available nurse based upon, for example, the location of that nurse with respect to that of the patient. Upon completion of administering the medication, the nurse provides data (e.g. feedback) of the visit, including an indication of any medication that was not taken and why. In some embodiments, if any medication was not taken, information regarding such is forwarded to the physician that prescribed that medication for remedial action.

In one embodiment, a system for mobile administration of medication includes a portable device for accessing the system by a nurse. The portable device has a global positioning subsystem. A server has scheduled visits for the nurse and each scheduled visit has a location for administration of the medication and a patient name. An application running on the portable device retrieves the set of scheduled visits from the server and presents one of the scheduled visits on a display of the portable device. At the location for administration of the medication, the application accepts an indication from the nurse that indicates that an encounter has begun, then after administration of the medication is complete, the application accepts an indication from the nurse that indicates that the encounter has completed along with data regarding the administration of the medication. In some embodiments, a payment method established by the patent is debited by a fixed amount and a payment is made to the nurse.

In another embodiment, a method of mobile administration of medication is disclosed including indicating, by a nurse, that the nurse is willing to accept unscheduled visit request, then until indicating by the nurse that the nurse is no longer willing to accept unscheduled visit request: if an unscheduled visit aligns with the nurse (e.g. is within a certain distance or area of the patient), the nurse receives a patient record for the unscheduled visit that includes a location of the patient and the nurse travels to a the location of the patient for administration of the medication(s) and the nurse indicates that the encounter has begun. The nurse then administers the medication, then, after administering the medication, the nurse indicates that the encounter has completed along with providing data regarding the administration of the medication. In some embodiments, a fixed amount is then debited from a payment method established by the patent and a payment is distributed to the nurse.

In another embodiment, a method of mobile administration of medication is disclosed including indicating by a nurse that the nurse is willing to accept unscheduled visit request. Now, until indicating by the nurse that the nurse is no longer willing to accept unscheduled visit request: it is determined if there is an unscheduled visit and that the unscheduled visit aligns with the nurse and if so: the nurse receives a patient record for the unscheduled visit that includes a location of the patient and then the nurse travels to a the location of the patient for administration of the medication(s). The nurse then indicates that an encounter has begun and the nurse administering the medication(s) to the patient. After administering the medication(s), the nurse indicates that the encounter has completed along with providing data regarding the administration of the medication(s).

### BRIEF DESCRIPTION OF THE DRAWINGS

The invention can be best understood by those having ordinary skill in the art by reference to the following detailed description when considered in conjunction with the accompanying drawings in which:

FIG. 1 illustrates a data connection diagram of the system for a mobile medication nurse.

FIG. 2 illustrates a schematic view of a typical cell phone.

FIG. 3 illustrates a schematic view of a typical computer system such as a server or personal computer.

FIG. 4 illustrates an exemplary cell phone user interface of the system for a mobile medication nurse showing an example of a reminder/acknowledgement of self-medication.

FIG. 5 illustrates an audio user interface of the system for a mobile medication nurse showing an example of a reminder/acknowledgement of self-medication.

FIG. 6 illustrates a user interface of the system for a mobile medication nurse showing selection of a primary medication administrator for assisted medication.

FIG. 7 illustrates a user interface of the system for a mobile medication nurse showing details of a primary interface for the medication administrator.

FIG. 8 illustrates a user interface of the system for a mobile medication nurse showing scheduled patients.

FIG. 9 illustrates a user interface of the system for a mobile medication nurse showing details of an appointment.

FIG. 10 illustrates a user interface of the system for a mobile medication nurse showing a notice that a patient requires an unexpected visit to administer medication.

FIG. 11 illustrates a user interface of the system for a mobile medication nurse showing patient information and an initialization step.

FIG. 11A illustrates a user interface of the system for a mobile medication nurse showing a way for the nurse to signal completion of the encounter.

FIGS. 12-14 illustrate user interfaces of the system for a mobile medication nurse reviewing an encounter with the patient.

FIGS. 15, 16, and 17 illustrate exemplary program flows of the system for a mobile medication nurse.

### DETAILED DESCRIPTION

Reference will now be made in detail to the presently preferred embodiments of the invention, examples of which are illustrated in the accompanying drawings. Throughout the following detailed description, the same reference numerals refer to the same elements in all figures.

In general, the medication nurse management system provides capabilities to manage a nursing staff that administers medications to patients. At least two types of patient encounters are expected; scheduled visits that are pre-scheduled and unscheduled visits that are needed when an it is determined that a patient, for some reason, failed to acknowledge self-administration of a medication.

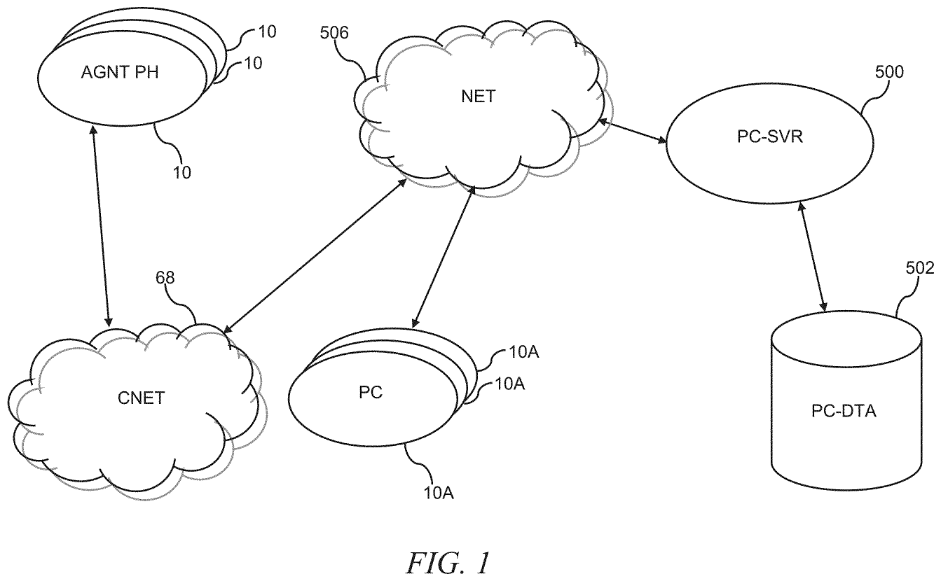

Referring to FIG. 1 illustrates a data connection diagram of the exemplary mobile medication nurse management system. In this example, one or more cell phones 10 communicate through the cellular network 68 and/or through a wide area network 506 (e.g. the Internet) to a server computer 500. In some embodiments, user computer systems 10A also communicate through the cellular network 68 and/or through a wide area network 506 (e.g. the Internet) to the server computer 500.

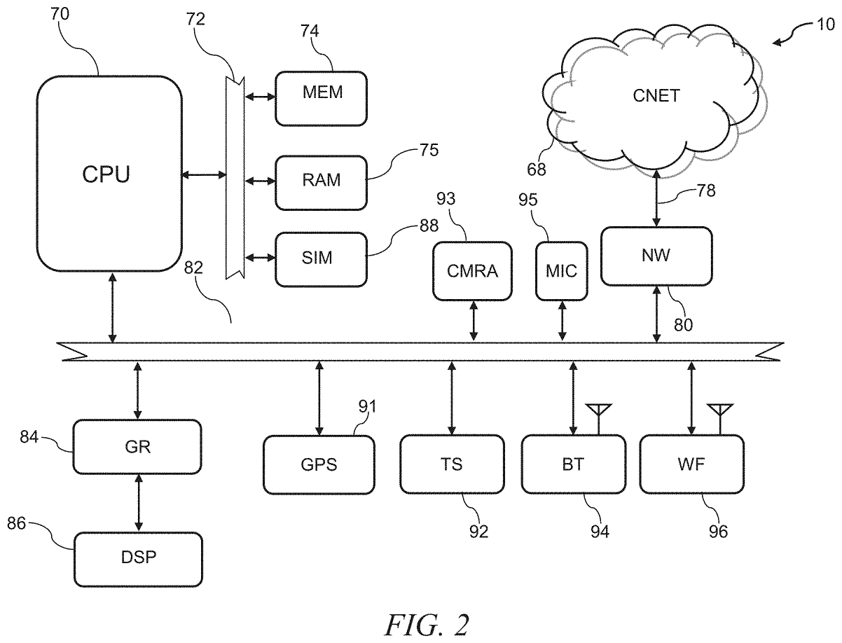

The server computer 500 has access to data storage 502. Although one path between the cell phones 10 and the server 500 is through the cellular network 68 and the wide area network 506 as shown, any known data path is anticipated. For example, the Wi-Fi transceiver 96 (see FIG. 2) of the cell phone 10 is used to communicate directly with the wide area network 506, which includes the Internet, and, consequently, with the server computer 500.

The server computer 500 transacts with the cell phones 10 through the network(s) 68/506 to present menus to/on the cell phones 10 (e.g. through a browser or dedicated application), provide data to the cell phones 10, and to communicate information such as nurse location, etc. In some embodiments, login credentials (e.g., passwords, pins, secret codes) are stored local to the cell phone 10; while in other embodiments, login credentials are stored in a data storage 502 (preferably in a secured area) requiring a connection to login.

The server computer 500 transacts with applications running on the cell phones 10 and/or with applications (e.g., browsers) running on the user computers 10A.

In some embodiments, the geographic area of the cell phone 10 is determined by reading the GPS subsystem 91 (see FIG. 2) of the cell phone 10 or by manual entry by a user. Manual entry by a user is made by, for example, entering a street address or by selecting a location on a map.

In some embodiments, the location from the GPS subsystem 91 is forwarded to the server 500 periodically for nurse tracking and/or unscheduled visit scheduling.

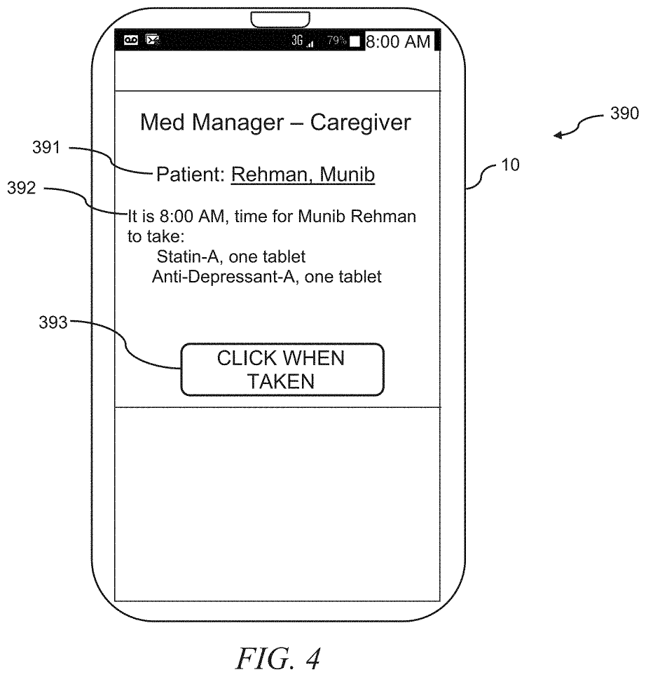

Referring to FIG. 2, a schematic view of a typical cell phone 10 is shown. The example cell phone 10 represents a typical phone system used for accessing user interfaces (see FIGS. 4-13) of the system for mobile medication nurse management. This exemplary cell phone 10 is shown in its simplest form. Different architectures are known that accomplish similar results in a similar fashion and the present invention is not limited in any way to any particular cell phone 10 system architecture or implementation. In this exemplary cell phone 10, a processor 70 executes or runs programs in a random access memory 75. The programs are generally stored within a persistent memory 74 and loaded into the random access memory 75 when needed. Also accessible by the processor 70 is a SIM (subscriber information module) card 88 having a subscriber identification and often persistent storage. The processor 70 is any processor, typically a processor designed for phones. The persistent memory 74, random access memory 75, and SIM card are connected to the processor by, for example, a memory bus 72. The random access memory 75 is any memory suitable for connection and operation with the selected processor 70, such as SRAM, DRAM, SDRAM, RDRAM, DDR, DDR-2, etc. The persistent memory 74 is any type, configuration, capacity of memory suitable for persistently storing data, for example, flash memory, read only memory, battery-backed memory, magnetic memory, etc. In some exemplary cell phones 10, the persistent memory 74 is removable, in the form of a memory card of appropriate format such as SD (secure digital) cards, micro SD cards, compact flash, etc.

Also connected to the processor 70 is a system bus 82 for connecting to peripheral subsystems such as a cellular network interface 80, a graphics adapter 84 and a touch screen interface 92. The graphics adapter 84 receives commands from the processor 70 and controls what is depicted on a display image on the display 86. The touch screen interface 92 provides navigation and selection features.

In general, some portion of the persistent memory 74 and/or the SIM card 88 is used to store programs, executable code, phone numbers, contacts, and data, etc. In some embodiments, other data is stored in the persistent memory 74 such as audio files, video files, text messages, etc.

The peripherals are examples and other devices are known in the industry such as Global Positioning Subsystem 91, speakers, microphones, USB interfaces, Bluetooth transceiver 94, Wi-Fi transceiver 96, camera 93, microphone 95, image sensors, temperature sensors, etc., the details of which are not shown for brevity and clarity reasons.

The cellular network interface 80 connects the cell phone 10 to the cellular network 68 through any cellular band and cellular protocol such as GSM, TDMA, LTE, etc., through a wireless medium 78. There is no limitation on the type of cellular connection used. The cellular network interface 80 provides voice call, data, and messaging services to the cell phone 10 through the cellular network.

For local communications, many cell phones 10 include a Bluetooth transceiver 94, a Wi-Fi transceiver 96, or both. Such features of cell phones 10 provide data communications between the cell phones 10 and data access points and/or other computers such as a personal computer (not shown).

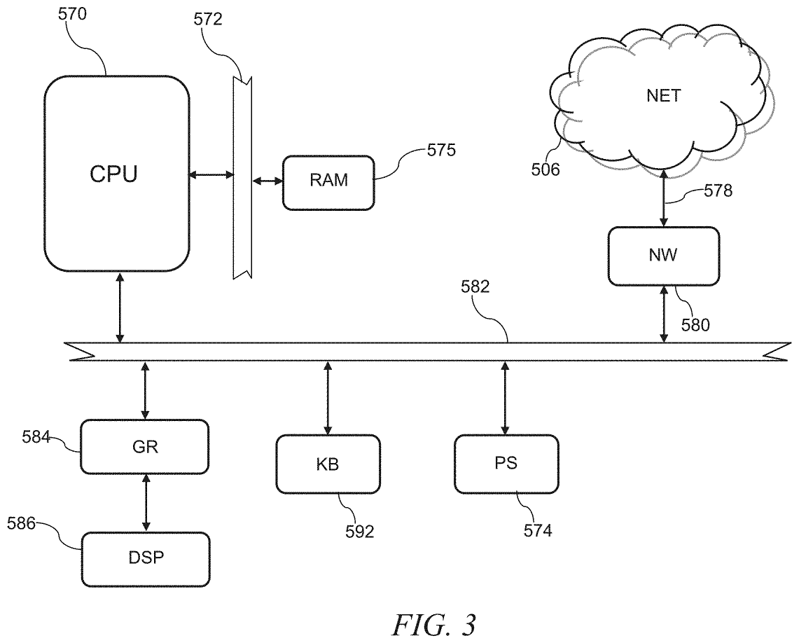

Referring to FIG. 3, a schematic view of a typical computer system (e.g., server 500 or user computer 10A) is shown. The example computer system 500 represents a typical computer system used for back-end processing, generating reports, displaying data, etc. This exemplary computer system is shown in its simplest form. Different architectures are known that accomplish similar results in a similar fashion and the present invention is not limited in any way to any particular computer system architecture or implementation. In this exemplary computer system, a processor 570 executes or runs programs in a random access memory 575. The programs are generally stored within a persistent memory 574 and loaded into the random access memory 575 when needed. The processor 570 is any processor, typically a processor designed for computer systems with any number of core processing elements, etc. The random access memory 575 is connected to the processor by, for example, a memory bus 572. The random access memory 575 is any memory suitable for connection and operation with the selected processor 570, such as SRAM, DRAM, SDRAM, RDRAM, DDR, DDR-2, etc. The persistent memory 574 is any type, configuration, capacity of memory suitable for persistently storing data, for example, magnetic storage, flash memory, read only memory, battery-backed memory, magnetic memory, etc. The persistent memory 574 is typically interfaced to the processor 570 through a system bus 582, or any other interface as known in the industry.

Also shown connected to the processor 570 through the system bus 582 is a network interface 580 (e.g., for connecting to a data network 506), a graphics adapter 584 and a keyboard interface 592 (e.g., Universal Serial Bus—USB). The graphics adapter 584 receives commands from the processor 570 and controls what is depicted on a display image on the display 586. The keyboard interface 592 provides navigation, data entry, and selection features.

In general, some portion of the persistent memory 574 is used to store programs, executable code, data, contacts, and other data, etc.

The peripherals are examples and other devices are known in the industry such as speakers, microphones, USB interfaces, Bluetooth transceivers, Wi-Fi transceivers, image sensors, temperature sensors, etc., the details of which are not shown for brevity and clarity reasons.

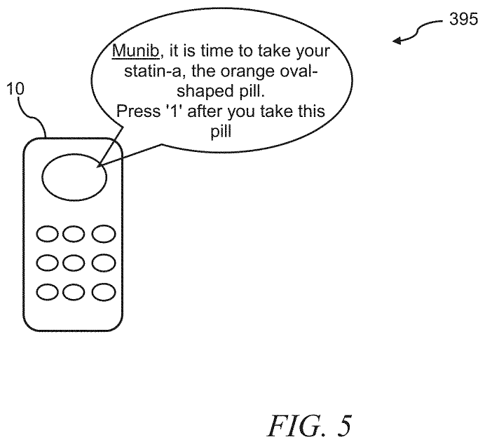

Referring to FIGS. 4 and 5, exemplary user interfaces for confirming a medication was taken when expected are shown. These user interfaces request that the patient acknowledge that the medication was taken. When the patient 391 is scheduled to take the medication, the patient 391 is notified by, for example, a text message, a pop-up message 390 from an application, or a voice call 395. Many patients 391 have medical or aging issues that make it easy to miss taking their medications when required.

In FIG. 4, the name of the patient 391 is displayed along with a list of medications 392 that are to be taken at a given time. A directive 393 is provided for the patient 391 to acknowledge that they have taken their medication. If the patient indicates that they took these medications (e.g. by invoking the “Click when Taken” directive 393) it is assumed that the patent has self-administered. If, after a specific amount of time, there is no acknowledgement from the patient 391 (the patient 391 did not invoke the directive 393), escalation procedures begin. In some embodiments, the patient 391 is given a dispensing device such as a pill box and that device records access events and is connected to a computer/communications device such that when the patient 391 accesses the dispensing device, a transaction is sent to the server indicating such and, it is assumed, that therefore, the patient has taken the medication associated with the dispensing device. As with the application described above, if a period of time lapses (e.g. 30 minutes) and the patient has not accessed the dispensing device, a warning message is sent to the patient. After another period of time lapses (e.g. 30 minutes) and still, the patient has not accessed the dispensing device, escalation procedures begin.

As will be shown, the escalation procedures include dispatching a nurse to check on the patient and administer the medication(s).

In FIG. 5, another exemplary method of informing a patient 391 that it is time to take their medication is shown. In this example, a voice call is made to the smartphone 10 (or any phone associated with the patient 391) and upon answering, a first audio message 395 is played, coming out of the smartphone's 10 earpiece or speaker saying that it is time to take the statin-A pill and instructing the patient 401 to “press ‘1’” after taking the statin-A pill. After the patient presses ‘1’, if there are additional medications that need be taken at the same time, subsequent audio message are played as needed. After all medications have been acknowledged, it is assumed that the patent has self-administered. If the patient 391 does not answer the phone call or if after a specific amount of time there is no acknowledgement from the patient 391 (the patient 391 did press the required key), escalation procedures begin.

It is fully anticipated that a similar user interface be presented on a television (e.g. smart TV), perhaps using the audio portion of the television to emit the audio message and using the television remote control to confirm that the medication was taken (e.g. press the ‘1’ on your remote control). Likewise, in some embodiments, the request to take the medication is displayed in text on the television instead of audio.

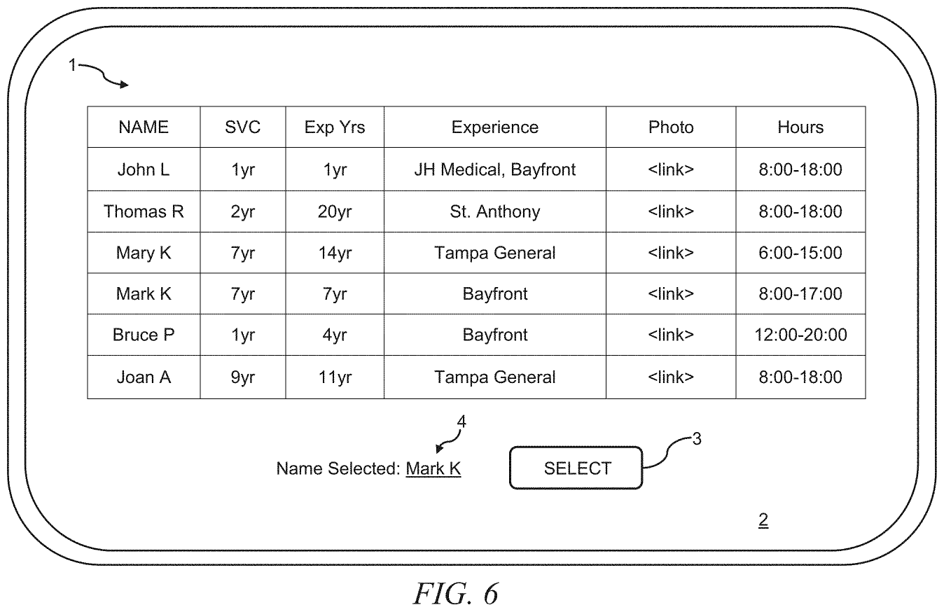

Referring to FIG. 6, a user interface 2 of the system for a mobile medication nurse showing selection of a primary medication administrator for assisted medication is shown. For certain patients, scheduled visits are required as such patients often have difficulty taking their medications at the prescribed time, for example, due to lack of memory or illness. Further, some medications require administration by a nurse, for example, those administered through an injection. In such, it is preferred that, as much as possible, the same nurse will visit the patient each day at the time when the medication is to be administered. Note, as with most medical systems, there are security measures for protecting staff and information (e.g. login credentials) and creation of accounts and entering of such has been left out for clarity and brevity reasons.

Therefore, once the patient (or patient with advocate) signs up for the mobile medication nurse service and requests scheduled visits, the patient and/or advocate is presented with a selection screen 1 showing the available nurses and data regarding those nurses such as each nurses years with mobile medication nurse service, total years being a nurse, experience (previous employers or types of employment), a link to a photograph, and hours in which each nurse is available for administering medications, etc. Once the patient/advocate selects a nurse, the name of the nurse selected appears in a field 4 and, once ready to commit to that nurse, the patient/advocate completes the selection by clicking on the “select” button.

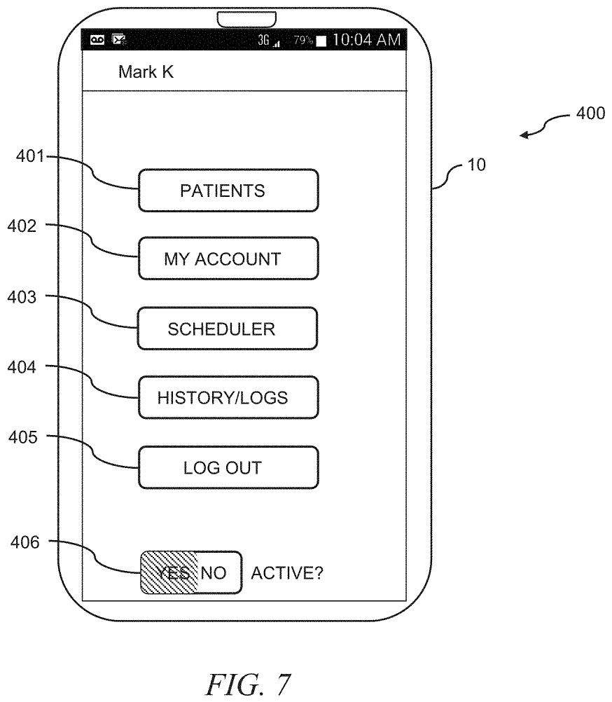

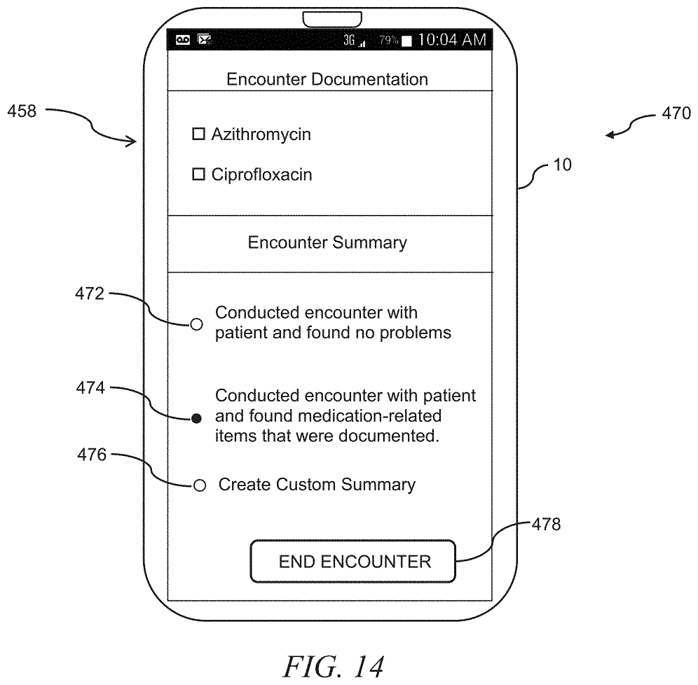

FIGS. 7-14 show exemplary user interfaces of the system for mobile medication nurse management as used by the nurse. The primary user interface 400 shown in FIG. 7 provides for navigation to the nurse's patients 401, to the nurse's account 402, to the nurse's scheduler 403 (for scheduled visits), to view the nurse's history 404, and to log out 405.

As discussed above, there is a need to make unscheduled visits to patients who are self-administering medications and, for some reason, do not acknowledge having taken their medication. For example, if one of the user interfaces shown in FIGS. 4 and 5 are used and the patient does not acknowledge having taken their medication for half an hour, and maybe for an additional half an hour if a missed-medication reminder is sent, the mobile medication nurse system will allocate a nurse that will visit that patient and make sure the patient is well and takes their medication. To accomplish this, a pool of nurses is maintained, including nurses that provide scheduled visits and/or nurses that have no scheduled visits. When a nurse is available for unscheduled visits, the nurse changes their active status 406 to “Yes.” When the nurse is no longer active (e.g. finished working, having lunch, etc.) the nurse changes their active status 406 to “No,” and that nurse will not be dispatched.

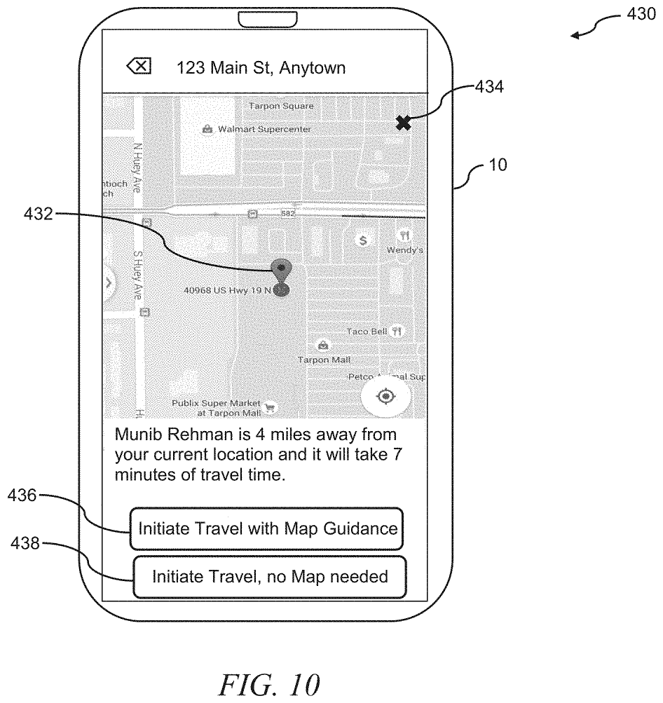

When it is declared that a patient needs an unscheduled visit, a list of nurses that are active and are in a specific geographic area related to the patient are considered for the unscheduled visit and one nurse is selected, for example, based upon that nurse's current location with respect to the patient, a least-recently used algorithm, or any other selection criteria. That nurse is then notified with the patient information as shown in FIG. 10.

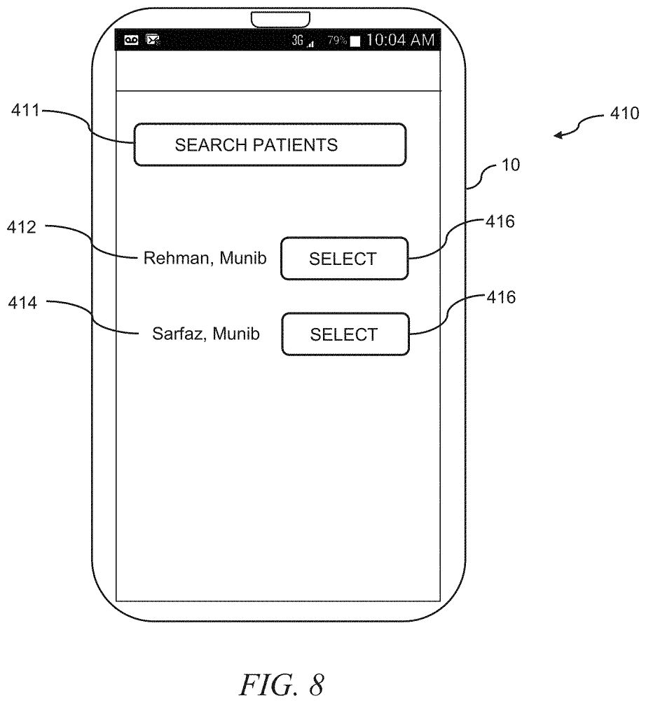

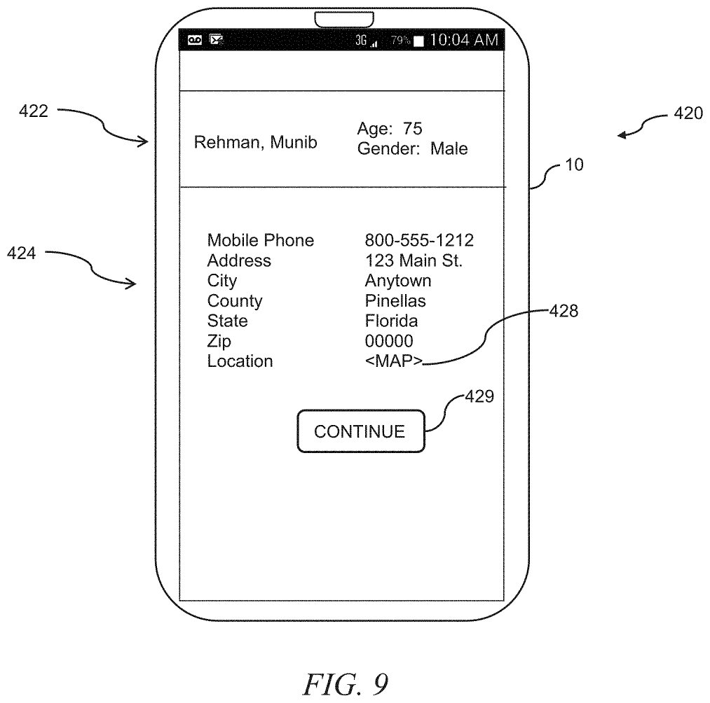

In FIG. 8, the nurse has selected patients and is presented with a list 410 of scheduled patients 412/414 for that nurse. For longer lists, the nurse has a feature for searching patients 411. To see details, the nurse selects a select icon 416 which will result in the user interface of FIG. 9. It is fully anticipated that the nurse utilize a scheduler built into the system for mobile medication nurse management or use any other scheduling practice, including a paper schedule, selecting patients as they appear on whatever scheduling practice is utilized.

In FIG. 9, the nurse is presented with details 420 of the selected patient such as name/age/gender 422 and location information 424. The location information, in some embodiments, includes a link to a map 428 that will show the nurse the location of the patient (similar to that shown in FIG. 10). When the nurse is ready, the nurse invokes “continue” 429.

In FIG. 10, the nurse has indicated that they are available for unscheduled visits and a patient has not acknowledged taking of their medications (e.g. an hour has passed or similar). The nurse, having set their status to active=yes, receives notice 430 of the unscheduled visit showing the nurse's location 434, the patient's location 432, an estimate of distance and travel time, and the ability to initiate travel with map guidance 436 or without map guidance 438.

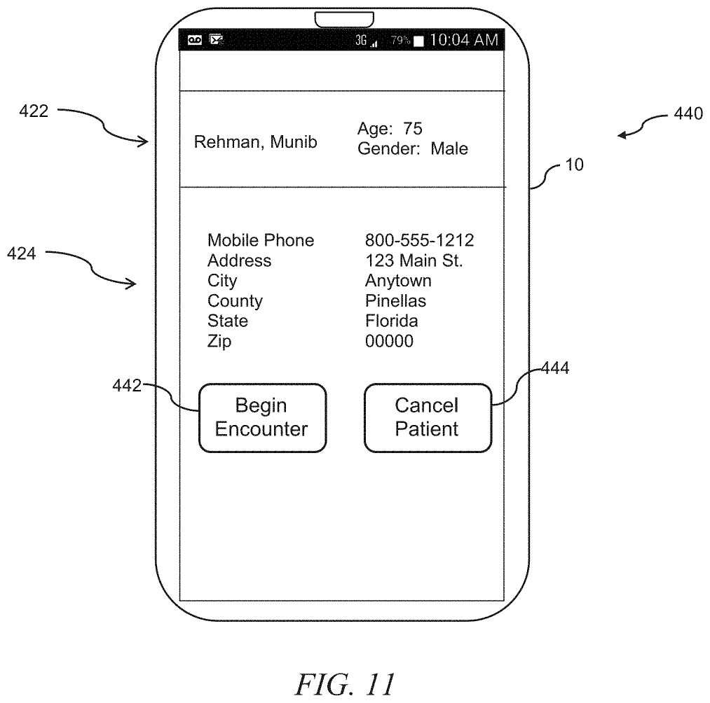

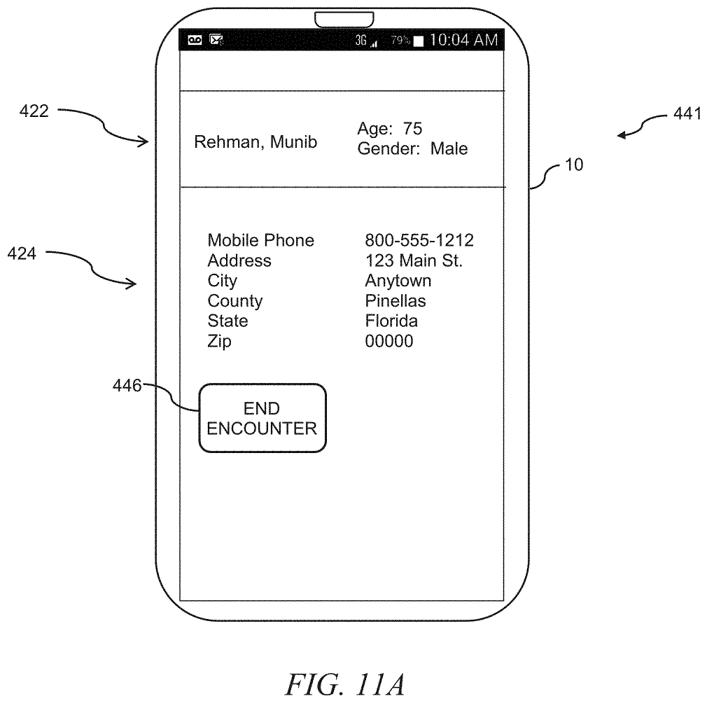

Whether an unscheduled visit or a scheduled visit, the nurse will be provided with an informational user interface 440 of FIG. 11 and has the ability to begin the patient encounter 442 or cancel the patient encounter 444. During the visit, a user interface of the system for a mobile medication nurse showing a way for the nurse to signal completion of the encounter 441 is provided including a “end encounter” 446 directive (e.g. medication had been administered or an issue has occurred).

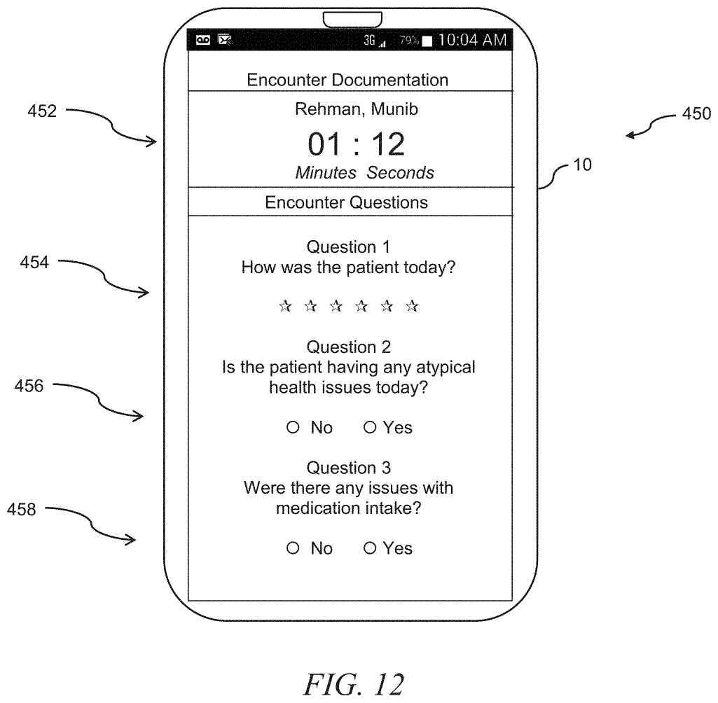

After the encounter, the nurse will receive a documentation user interface 450/460/470 as in FIGS. 12, 13, and 14. The first documentation user interface 450 shows the elapsed time 452 with the patient and three questions, though any number of questions are anticipated, including zero.

A first question 454 asks how the patient was. In this, the nurse rates the patient with a star rating (e.g. the better the patient, the more stars). The second question 456 asks if the patient is having any atypical health issues (yes/no) and the third question 458 asks if there were any issues with medication intake.

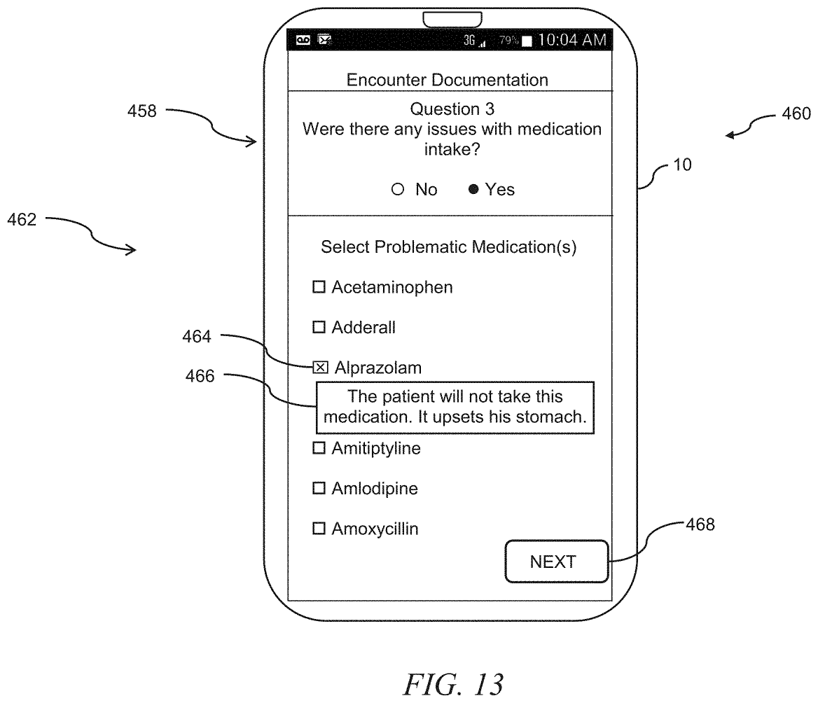

In this example, the nurse indicated that there were issues taking medications (third question 458) and the interface of FIG. 13 is presented listing the medications 462. In this, the nurse has checked a box for Alprazolam 464 and then entered text stating that this medication upsets the patient's stomach.

In the last documentation user interface 470, the nurse selects no problems 472, medication-related problems 474, or custom summary 476, then ends the encounter by selecting “end encounter” 478.

As can be expected, payment must be made for the services described. It is anticipated that a fixed fee be charge per visit. During the registration process, one or more forms of payment are registered and, after registration, it is anticipated that the patient/advocate is able to change or update such forms of payment. In some embodiments, payment is made through a credit card, through a debit card, by direct payment from a bank, or through a payment service. In some embodiments, payment is made in advance and a running account is maintained.

Each time a nurse completes an encounter, a billing record is made indicating the patient and the nurse. Either periodically (e.g. every Thursday night) or as the records are received, payments are debited from the form of payment of the patient (e.g. after each visit, a fixed amount such as $8.00 is charged to the credit card on record for the patient). Periodically (e.g. every Thursday night), payment to each nurse having made at least one visit is made as a fixed amount per visit (e.g. a nurse who made three visits during the week receives payment for three visits such as $18.00), by any payment system available including a paycheck, e-check, direct deposit, etc.

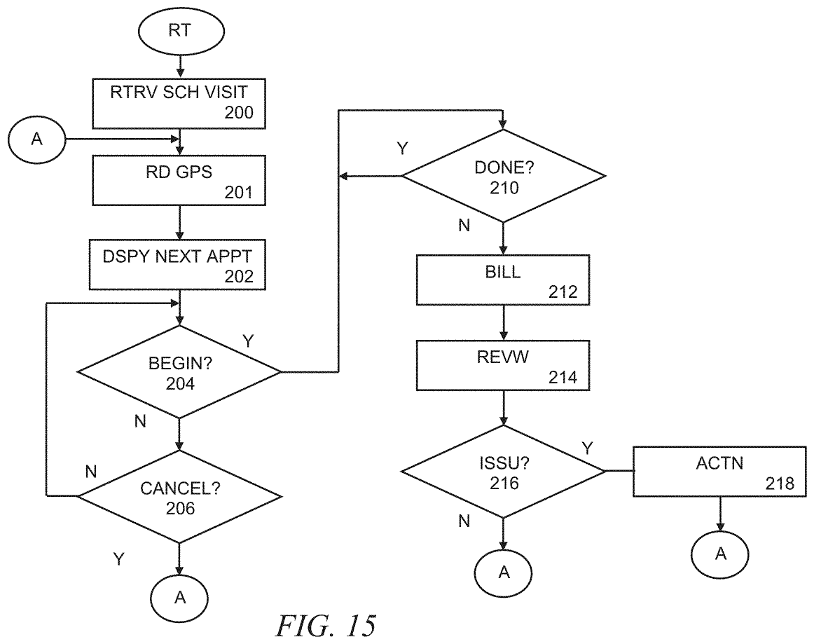

Referring to FIGS. 15, 16, and 17, exemplary program flows of the system for a mobile medication nurse are shown. It is anticipated that portions of the exemplary program flow execute on a user device such as a cell phone 10 and/or a personal computer 10A (e.g., using a browser or application running on the personal computer 10A) while portions of the exemplary program flow execute on the server 500.

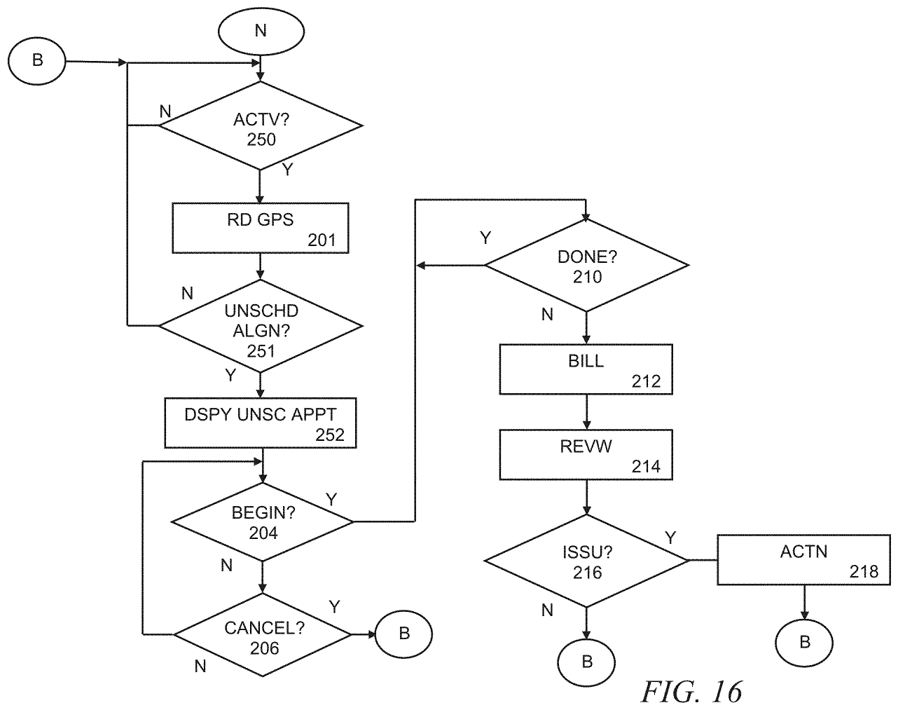

In this example, the flow starts retrieving 200 the list of scheduled visitation for administration of medications by this nurse. The Global Positioning System (GPS) 93 is consulted 201 to learn the location of the nurse and the next scheduled visitation is displayed 202 (e.g. with a map similar to that shown in FIG. 10). For brevity reasons, as is shown in FIG. 16, if the nurse has no upcoming scheduled visits, the nurse can declare themselves “active” 406 (see FIG. 7) and accept unscheduled visits.

Next, the nurse either indicates that he/she has begun the encounter 204 or canceled 206 the encounter. If canceled 206, flow reverts to reading the GPS 201 again and displaying the next appointment 202.

If the nurse indicates he/she has begun the encounter 204, the flow waits 210 for an indication that the encounter is complete by “end encounter” 446 (see FIG. 11A). At that point, a billing record 212 is recorded. Next encounter review data is retrieved 214 from the nurse (e.g. as described in FIGS. 12-14).

If an issue has been recorded 216, an action 218 is taken. In some embodiments, the action is to add notes to records related to the patient (e.g. “the patient had difficulty swallowing”). In some embodiments, for example when there are issues with medication administration, the prescriber of the medication is notified by, for example, a mailed letter, a fax, an email, direct connection, etc. An example of such is that the patient refuses to take a specific medication because it causes side effects (e.g. stomach ache). In this, it is often important to notify the prescriber (e.g. doctor) so that the prescriber knows that the medication is not being taken and the prescriber is able to prescribe a different medication or another way to reduce the side effects.

FIG. 16 describes the operation regarding unscheduled visits. In this example, the flow starts with checking 250 to see if the nurse is active (e.g., the active status 406 is set to “Yes”). Checking 250 loops until it is determined that the nurse active at which time it is determined 251 if there are any unscheduled visits that need to be made that align with the nurse. Now the GPS is read 201 (e.g. the GPS of a smartphone 10 of the nurse) and a test for unscheduled visits that align to the nurse is made 251. Note that there are many considerations anticipated in determining alignment. For example, an unscheduled visit aligns with the nurse if the nurse is within a distance range of the patient (e.g. within 10 miles), within the same town as the patient, within the same zip code as the patient (either 5 digit or 8 digit zip code), by driving time, etc. Further, if two or more nurses align with the same unscheduled visit (e.g. two or more nurses are active and local to the patient), a secondary algorithm is utilized to fairly allocate unsolicited visits. Such algorithms includes, for example, first to accept, a bidding algorithm (each nurse bids on the unscheduled visit and the lowest bid receives the unscheduled visit), a determination of which nurse is closest to the patient, an income averaging (e.g. the nurse who has received the least number of unscheduled visits gets this unscheduled visit), etc.

Whichever nurse is awarded the unscheduled visit, the unscheduled visit is displayed 252 (e.g. with a map similar to that shown in FIG. 10).

Next, as above, the nurse either indicates that he/she has begun the encounter 204 or canceled the encounter 206. If canceled 206, flow reverts to reading the GPS 201 again and displaying the next appointment 202.

If the nurse indicates he/she has begun the encounter 204, the flow waits 210 for an indication that the encounter is complete by “end encounter” 446 (see FIG. 11A). At that point, a billing record 212 is recorded. Next encounter review data is retrieved 214 from the nurse (e.g. as described in FIGS. 12-14).

If an issue has been recorded 216, an action 218 is taken. In some embodiments, the action is to add notes to records related to the patient (e.g. “the patient had difficulty swallowing”). In some embodiments, for example when there are issues with medication administration, the prescriber of the medication is notified by, for example, a mailed letter, a fax, an email, direct connection, etc. An example of such is that the patient refuses to take a specific medication because it causes side effects (e.g. stomach ache). In this, it is often important to notify the prescriber (e.g. doctor) so that the prescriber knows that the medication is not being taken and the prescriber is able to prescribe a different medication or another way to reduce the side effects.

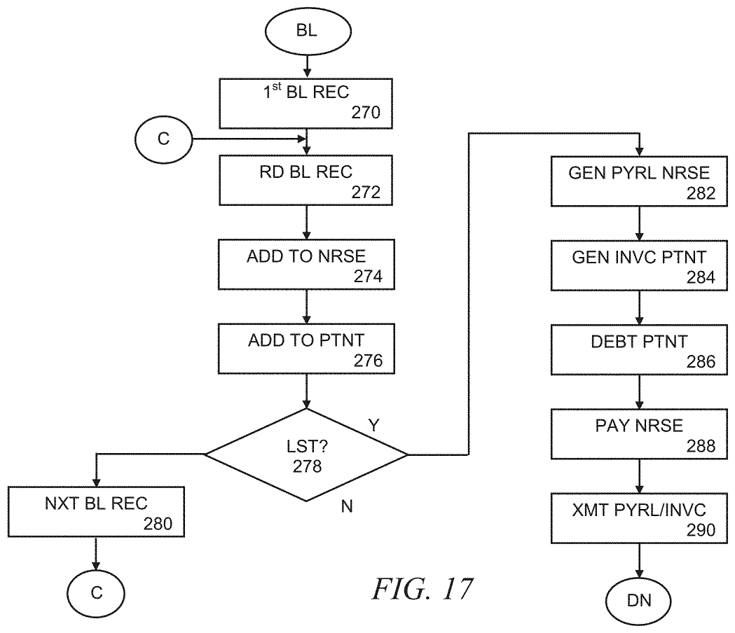

In FIG. 17, an exemplary financial system is shown for debiting the patient and paying the nurses. As discussed above, it is anticipated that each visit (either scheduled or unscheduled) the patient's form of payment is debited a fixed amount for the visit. It is anticipated that this activity is done each time the visit occurs, possibly leading to multiple debits per period, or once per period, leading to a single debit per period. The example shown in FIG. 17 performs debiting one time for the collection of visits for each patient. In this, the first billing record is addressed 270 (e.g., first billing record for this period). Note, any billing period is anticipated such as one week, one month, etc.

The addressed billing record is read 272 and an amount is added to the nurse's total 274 and another amount is added to the patient's total 276, along with any date and identifying information (e.g. Nurse Mark K visited on September 4^{th}). If this is not the last billing record 278, the next billing record is addressed 280 and the above steps repeat.

If this is the last billing record 278, the payroll for each nurse 282 and an invoice for each patient are generated 284. Now the payment is debited 286 from the form of payment selected by the patient and payment is made to each nurse 288 (e.g. paycheck, direct deposit). Finally, the payroll for each nurse and the invoice for each patient are transmitted 290 (e.g. sent by paper mail, email, text message, available for view on a web site).

Equivalent elements can be substituted for the ones set forth above such that they perform in substantially the same manner in substantially the same way for achieving substantially the same result.

It is believed that the system and method as described and many of its attendant advantages will be understood by the foregoing description. It is also believed that it will be apparent that various changes may be made in the form, construction and arrangement of the components thereof without departing from the scope and spirit of the invention or without sacrificing all of its material advantages. The form herein before described being merely exemplary and explanatory embodiment thereof. It is the intention of the following claims to encompass and include such changes.

What is claimed is:

1. A system for mobile administration of medication(s), the system comprising:
a portable device for accessing the system by a nurse, the portable device having a global positioning subsystem;
a server, the server having patient records for each of a plurality of patients, each record includes a list of medications and a location each patient of the plurality of patient;
an application running on the portable device retrieves one record of the plurality of patient records;
at the location of the patient, the application accepts an indication from the nurse that indicates that an encounter has begun; and
after administration of the medication(s) is complete, the application accepts a second indication from the nurse that indicates that the encounter has completed along with data regarding the administration of the medication(s).

2. The system of claim 1, whereas if the data regarding the administration of the medication(s) indicates a problem with a medication, a notice is transmitted to a physician that prescribed that medication.

3. The system of claim 1, further comprising:
after administration of the medication(s) is complete, a payment method established with the patient is debited by a fixed amount; and
a payment is made to the nurse.

4. The system of claim 1, further comprising:
the application running on the portable device receives a control from the nurse indicating that the nurse is willing to accept unscheduled visit request;
until the application running on the portable device receives a second control from the nurse indicating that the nurse is no longer willing to accept unscheduled visit request:
if an unscheduled visit aligns with the nurse:
the application running on the portable device receives a patient record for the unscheduled visit from the server and presents the patient record on a display of the portable device;
the nurse travels to the location of the patient;
at the location of the patient, the application accepts the indication from the nurse that indicates that the encounter has begun; and
after administration of the medication(s) is complete, the application accepts the second indication from the nurse that indicates that the encounter has completed along with data regarding the administration of the medication(s).

5. The system of claim 4, further comprising:
after administration of the medication(s) is complete, a payment method established with the patient is debited by a fixed amount; and
a payment is made to the nurse.

6. The system of claim 4, wherein the unscheduled visit aligns with the nurse if the nurse is within a certain range of the location of the patient.

7. The system of claim 4, wherein the unscheduled visit aligns with the nurse if the nurse is within a certain range of the location of the patient and the nurse is first to accept the unscheduled visit.

8. The system of claim 4, wherein the unscheduled visit aligns with the nurse if the nurse is within a same zip code in the location of the patient.

9. A method of mobile administration of medication, the method comprising:
indicating by a nurse that the nurse is willing to accept unscheduled visit request;
until indicating by the nurse that the nurse is no longer willing to accept unscheduled visit request:
determining if an unscheduled visit aligns with the nurse and if the unscheduled visit aligns with the nurse:
the nurse receiving a patient record for the unscheduled visit, the patient record including a location of the patient;
the nurse traveling to the location of the patient for administration of the medication(s);
the nurse indicating that an encounter has begun;
the nurse administering the medication(s) to the patient; and
after administering the medication(s) is done, the nurse indicating that the encounter has completed along with providing data regarding the administration of the medication(s).

10. The method of claim 9, further comprising:
debiting a fixed amount from a payment method established by the patient; and
distributing a payment to the nurse.

11. The method of claim 9, wherein the step of determining if the unscheduled visit aligns with the nurse includes determining if the nurse is within a certain range of the location of the patient.

12. The method of claim 9, wherein the step of determining if the unscheduled visit aligns with the nurse includes determining if the nurse is within a certain range of the location of the patient and the nurse is first to accept.

13. The method of claim 9, wherein the step of determining if the unscheduled visit aligns with the nurse includes determining if the nurse is within a certain range of the location of the patient and the nurse is a lowest bidder.

14. The method of claim 9, wherein the step of determining if the unscheduled visit aligns with the nurse includes determining if the nurse is within a same zip code in the location of the patient.

15. A method of mobile administration of medication, the method comprising:
indicating by a nurse at a smartphone that the nurse is willing to accept unscheduled visit request;
until indicating by the nurse at the smartphone that the nurse is no longer willing to accept unscheduled visit request:
determining if an unscheduled visit aligns with the nurse and if the unscheduled visit aligns with the nurse:
the smartphone of the nurse receiving and displaying a patient record for the unscheduled visit, the patient record including a location of the patient;
the nurse traveling to the location of the patient for administration of the medication(s);
the nurse indicating that an encounter has begun at the smartphone;
the nurse administering the medication(s) to the patient;
after administering the medication(s) is done, the nurse indicating at the smartphone that the encounter has completed along with providing data regarding the administration of the medication(s);
the smartphone communicating that the encounter has completed to a server; and
the server generating and storing a billing record.

16. The method of claim 15, further comprising:
after the nurse indicating at the smartphone that the encounter has completed, a billing record is stored at the server and the server periodically sorting the billing records by patient and debiting a fixed amount from a payment method established by the patient for each bill in the billing records; and
the server periodically sorting the billing records by nurse and distributing a payment to the nurse for each bill in the billing records.

17. The method of claim 15, wherein the step of determining if the unscheduled visit aligns with the nurse includes determining if the nurse is within a certain range of the location of the patient.

18. The method of claim 15, wherein the step of determining if the unscheduled visit aligns with the nurse includes determining if the nurse is within a certain range of the location of the patient and the nurse is first to accept.

19. The method of claim 15, wherein the step of determining if the unscheduled visit aligns with the nurse includes determining if the nurse is within a certain range of the location of the patient and the nurse is a lowest bidder.

20. The method of claim 15, wherein the step of determining if the unscheduled visit aligns with the nurse includes determining if the nurse is within a same zip code in the location of the patient.
